(19) United States
(12) Patent Application Publication
(10) Pub. No.: US 2026/0210925 A1
(43) Pub. Date: Jul. 23, 2026

(54) SYSTEM AND METHOD FOR ENHANCED PREDICTIVE FAILURE ANALYSIS OF NATURAL GAS EQUIPMENT USING AI-DRIVEN SENSOR AND EMISSIONS DATA INTEGRATION

(71) Applicant: Oasis Emission Consultants, Inc., Rock Springs, WY (US)

(72) Inventor: Gregory Hunter, Castle Rock, CO (US)

(73) Assignee: Oasis Emission Consultants, Inc., Rock Springs, WY (US)

(21) Appl. No.: 19/206,287

(22) Filed: May 13, 2025

Related U.S. Application Data
(63) Continuation-in-part of application No. 19/032,566, filed on Jan. 21, 2025, now Pat. No. 12,612,885.

(51) Int. Cl. G01N 33/00

(52) U.S. Cl. CPC G01N 33/0034; G01N 33/0037; G01N 33/004

(57) ABSTRACT

A system and method for capturing, monitoring, and utilizing fugitive combustible gases from natural gas compressors and engines. The system comprises collection points for capturing gases from multiple sources, a filtration system, a control valve, a programmable logic controller for monitoring and control, and an engine air intake system for utilizing the gases as supplementary fuel. Advanced AI-driven monitoring capabilities transform sensor and emissions data into predictive maintenance insights, enabling early detection of equipment failures through pattern analysis. The system integrates FTIR analyzers, electrochemical sensors, and SCADA connectivity to provide comprehensive emissions monitoring and regulatory compliance documentation while optimizing operational efficiency through probability-based failure predictions.

### CROSS REFERENCE TO RELATED APPLICATIONS

This application is a continuation-in-part of U.S. patent application Ser. No. 19/032,566, filed Jan. 21, 2025, entitled “SYSTEM AND METHOD FOR CAPTURING AND UTILIZING FUGITIVE COMBUSTIBLE GASES DURING NATURAL GAS PRODUCTION,” the entire contents of which are hereby incorporated by reference.

### FIELD OF THE INVENTION

The invention relates to systems and methods for capturing, monitoring, and utilizing fugitive combustible gases from natural gas production and compression facilities to reduce greenhouse gas emissions and improve operational efficiency.

### BACKGROUND OF THE INVENTION

The natural gas industry faces significant technical and regulatory challenges related to fugitive emissions, equipment reliability, and operational efficiency. U.S. patent application Ser. No. 19/032,566 addressed critical aspects of fugitive gas capture and utilization, providing an important foundation for emissions reduction and regulatory compliance. However, several interconnected technical problems remain unresolved by existing systems, such as the system described in the '566 application as referred to above.

Current natural gas equipment monitoring systems operate primarily in a reactive mode, detecting failures only after they have occurred or are imminent. These reactive approaches result in costly emergency shutdowns averaging $500,000+ per incident, unplanned emissions during equipment failure events, inefficient maintenance scheduling and resource allocation, and inability to predict and prevent cascading equipment failures. Previously disclosed art, while providing comprehensive data logging capabilities, lacks predictive analytical capabilities that could transform collected data into actionable maintenance insights before catastrophic failures occur.

Existing monitoring systems typically operate in silos, collecting various types of data without integration. Engine performance data remains isolated from emissions data, compressor operational metrics are analyzed separately from exhaust composition, packing vent flow measurements are not correlated with other operational parameters, and compliance testing data is collected episodically without integration into continuous monitoring. This fragmentation of data prevents operators from identifying complex failure patterns that manifest across multiple subsystems simultaneously. While other known art collects various operational parameters, it does not provide mechanisms for integrated analysis across these disconnected data streams.

A critical technical gap exists in correlating emissions signatures with specific equipment degradation patterns. Changes in exhaust gas composition often precede mechanical failures, subtle patterns in NOx, CO, O2, and CH4 emissions can signal specific degradation modes, and fluctuations in emissions combined with operational parameters provide early warning signs. Traditional monitoring systems analyze these data sources separately, missing crucial correlations.

Current approaches to regulatory compliance often treat emissions monitoring as a separate function from operational performance. Compliance testing provides point-in-time emissions data without operational context, Waste Emissions Charge (WEC) calculations focus on reporting without prevention, NSPS OOOO b/c compliance is managed separately from equipment maintenance, and emissions reduction efforts are not systematically linked to equipment reliability initiatives. The parent application addresses regulatory compliance comprehensively but does not fully integrate compliance functions with predictive operational insights.

Conventional monitoring systems utilize basic threshold alarms and trend analysis, but lack sophisticated intelligence. Complex patterns across multiple parameters are difficult to detect with conventional methods, subtle correlations between seemingly unrelated variables remain hidden, early warning signs involving multiple subtle changes go undetected, and the wealth of sensor and emissions data remains underutilized for predictive purposes. The parent application's data logging system captures extensive operational data but doesn't employ advanced analytical methods to extract complex predictive patterns from this data.

Existing approaches to these problems have significant limitations. Traditional SCADA systems monitor either operational parameters or emissions data, but rarely integrate both effectively. The parent application provides more comprehensive monitoring than traditional systems but still treats these data streams primarily as separate entities. Compliance testing using FTIR analyzers provides detailed emissions data, but only as periodic snapshots without integration into continuous monitoring systems. The parent application's comprehensive data logging doesn't specifically address integrating periodic compliance test data with continuous monitoring.

Skilled technicians manually review operational and emissions data to identify potential issues, an approach that is time-consuming and often misses subtle correlations. The parent application's HMI provides real-time data but relies on operator interpretation rather than automated intelligence. Conventional systems rely on simple threshold violations rather than complex pattern recognition across multiple parameters. The parent application includes sophisticated safety systems but primarily uses threshold-based approaches rather than pattern recognition. Engine analysis systems, emissions monitoring systems, and compressor monitoring systems typically operate as separate platforms with limited data sharing, creating analytical blind spots. The parent application provides a more integrated monitoring approach but doesn't specifically address cross-system pattern analysis.

The industry requires an innovative approach that combines fugitive gas capture with intelligent, integrated monitoring to provide truly predictive capabilities that can dramatically improve equipment reliability, regulatory compliance, and operational efficiency. The technical limitations of existing systems, including those addressed in the parent application, highlight the need for a novel combined approach that integrates fugitive gas capture with AI-driven monitoring. This integrated approach would transform reactive maintenance into truly predictive maintenance, connect previously siloed data streams for holistic analysis, correlate emissions signatures with specific equipment degradation patterns, integrate regulatory compliance with operational optimization, and apply artificial intelligence to extract complex predictive patterns from comprehensive data.

### SUMMARY OF THE INVENTION

In an embodiment of the invention, a system and method are provided that combine fugitive gas capture technology with artificial intelligence-driven monitoring capabilities for natural gas production equipment. This integrated approach, in a preferred embodiment, addresses critical industry challenges related to emissions reduction, equipment reliability, and regulatory compliance through a novel combination of gas capture mechanics and predictive analytics.

In an example of the invention, the system integrates a comprehensive fugitive gas capture system with sophisticated AI-driven failure prediction capabilities. The fugitive gas capture component, in a preferred embodiment, collects emissions from multiple sources including compressor cylinder packings, engine crankcases, instrumentation vents, gas dehydration units, and storage tanks. These captured gases, in an embodiment of the invention, undergo filtration and are reintroduced into the engine air intake system as supplementary fuel, simultaneously reducing emissions and improving operational efficiency.

The AI-driven monitoring capabilities, in a preferred embodiment of the invention, transform the system from a reactive emissions management tool into a predictive maintenance platform. By analyzing correlations between multiple data streams—including engine parameters, compressor operational metrics, and emissions signatures—an embodiment of the system can identify complex failure patterns before they result in equipment breakdown. For example, the AI model may analyze relationships between O2 fluctuations and manifold pressure instability to predict potential engine problems, or examine packing vent flow measurements alongside pressure readings to forecast possible compressor issues.

In an embodiment, the invention incorporates advanced analytics capabilities through several integrated components. MKS FTIR analyzers provide comprehensive exhaust monitoring of NOx, CH₄, CO, and O₂ concentrations. A compliance testing trailer, in a preferred embodiment of the invention, interfaces directly with the main system's PLC, enabling synchronized data collection during test operations. Optional stationary emission sensors, including electrochemical sensors for NOx, CO, and O₂ and NDIR sensors for CH₄, may be included in an embodiment to enable extended monitoring periods when concerning patterns emerge.

A key technical advantage of a preferred embodiment is the system's ability to generate specific probability-based failure predictions through its AI model, such as identifying a “65% chance of engine failure in 72 hours” based on analyzed data patterns. This predictive capability, in an example of the invention, reduces unplanned downtime, prevents environmental releases during failure events, and optimizes maintenance scheduling.

The invention, in an embodiment, further offers integration with facility-wide SCADA systems, allowing for automated data transmission at configurable intervals. This creates a comprehensive operational history for both immediate monitoring and long-term trend analysis.

By combining emissions capture with predictive analytics, an embodiment of the invention represents a significant technological advancement over systems that treat emissions monitoring and equipment reliability as separate functions. This integrated approach enables facilities to simultaneously address environmental compliance requirements and operational optimization goals while substantially reducing the financial impact of equipment failures and emissions penalties.

In a preferred embodiment, the system transforms disconnected data streams into integrated predictive insights by correlating emissions patterns with specific equipment degradation signatures, provides early failure detection through AI analysis of combined sensor and emissions data patterns, enables precise quantification of environmental impact and regulatory compliance through comprehensive data collection, optimizes maintenance scheduling through probability-based failure predictions, facilitates both emissions reduction and equipment reliability through a unified platform, and supports flexible deployment options with permanent or portable analyzer configurations.

In an example of the invention, the system offers particularly valuable applications in remote natural gas production facilities where equipment failures result in substantial financial losses and environmental impacts. By detecting subtle changes in operating parameters and emissions signatures before catastrophic failures occur, an embodiment of the system enables operators to address incipient issues during scheduled maintenance rather than through emergency response. Additionally, the integration with existing regulatory compliance workflows, in a preferred embodiment, streamlines reporting requirements while providing actionable operational insights that were previously unavailable through conventional monitoring approaches.

The implementation of this combined system, in an embodiment of the invention, provides immediate and long-term benefits, including reduced unplanned emissions, extended equipment life, optimized maintenance schedules, enhanced regulatory compliance documentation, and significant cost savings through failure prevention. These benefits derive directly from the novel technical approach of integrating fugitive gas capture with AI-driven predictive monitoring in a way that fundamentally transforms both processes.

### BRIEF DESCRIPTION OF THE FIGURES

FIG. 1 is a diagrammatic view of a greenhouse gas capture system according to an exemplary embodiment.

FIG. 2 is a diagrammatic illustration showing the collection points for capturing fugitive gases from multiple sources within a natural gas production facility according to an exemplary embodiment.

FIG. 3 is a detailed cross-sectional view of the filtration system showing the coalescing filter and associated components according to an exemplary embodiment.

FIG. 4 is a flowchart illustration of the control system showing the interaction between the PLC, sensors, and control components according to an exemplary embodiment.

FIG. 5 is a schematic diagram of the safety systems showing the primary and secondary pressure relief valves and associated safety components according to an exemplary embodiment.

FIG. 6 is a representation of the human-machine interface (HMI) screen showing system performance data and control options according to an exemplary embodiment.

FIG. 7 is a block diagram of the data logging and reporting system showing the flow of information from sensors to storage according to an exemplary embodiment.

FIG. 8 demonstrates the integration of the system components with existing engine and compressor equipment in a typical industrial environment according to an exemplary embodiment.

### DETAILED DESCRIPTION

The present invention relates to a system and method for capturing, monitoring, and utilizing fugitive combustible gases, primarily methane, from natural gas compressors and engines. This innovative system addresses the critical issue of greenhouse gas emissions in the oil and gas industry while simultaneously improving fuel efficiency and operational performance.

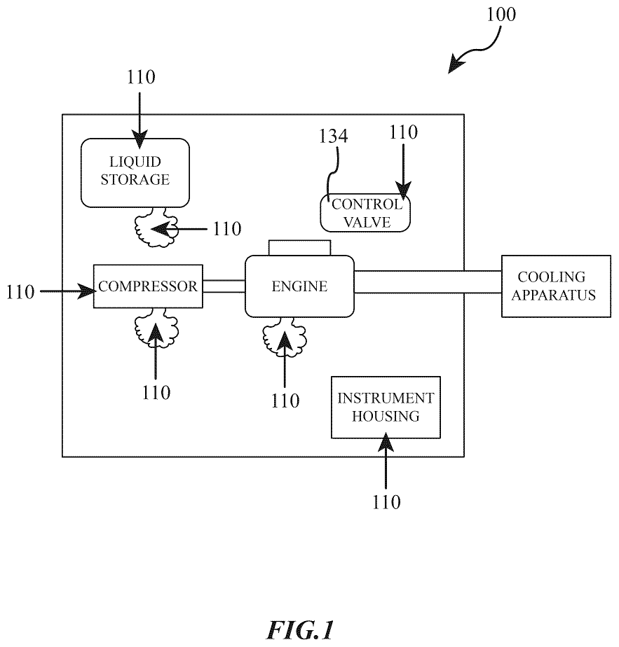

As shown in FIGS. 1-8, the preferred embodiment comprises an overall vent gas capture system 100 that includes multiple integrated subsystems working together to capture, process, and utilize fugitive gases. FIG. 1 illustrates the system overview in accordance with the preferred embodiment, showing strategically placed gas collection points 110 positioned throughout the facility to capture emissions from various sources including compressor packings, engine crankcases, and instrumentation vents.

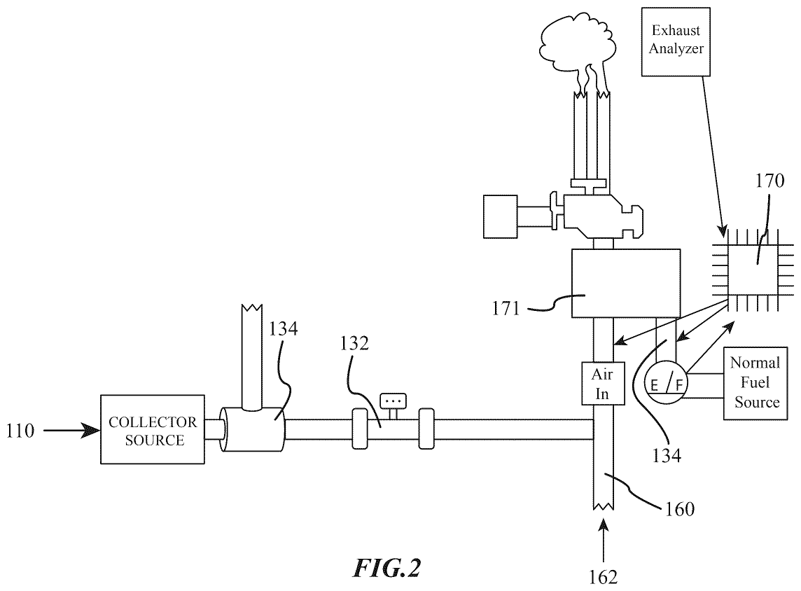

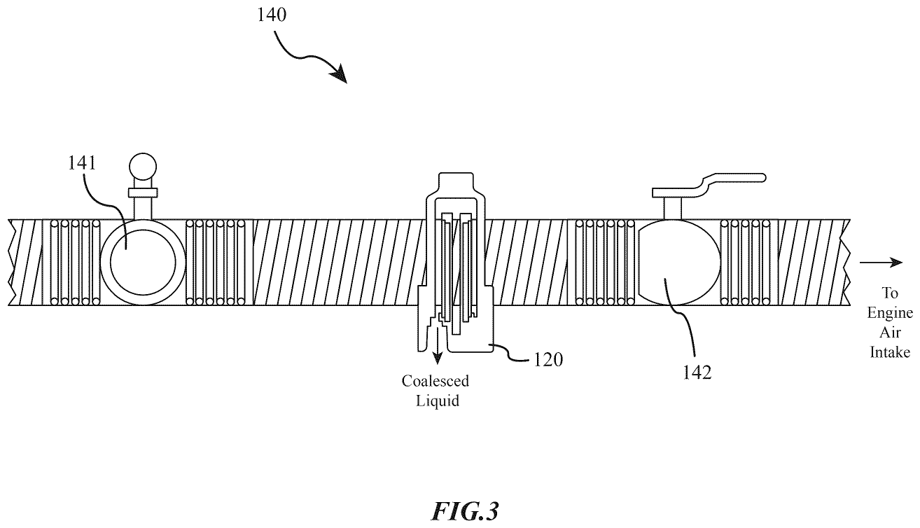

FIG. 2 depicts the filtration system 120 in accordance with the preferred embodiment which incorporates a coalescing filter designed to remove oil and other contaminants. The filtration system 120 includes isolation ball valves on either side to enable maintenance without system shutdown. The filtered gases then flow to the control system components 130, which include a programmable logic controller (PLC) 131, flow meters 132, pressure sensors 133, and control valves 134. FIG. 3 shows the detailed cross-section of the filtration system 120 in accordance with the preferred embodiment and its components.

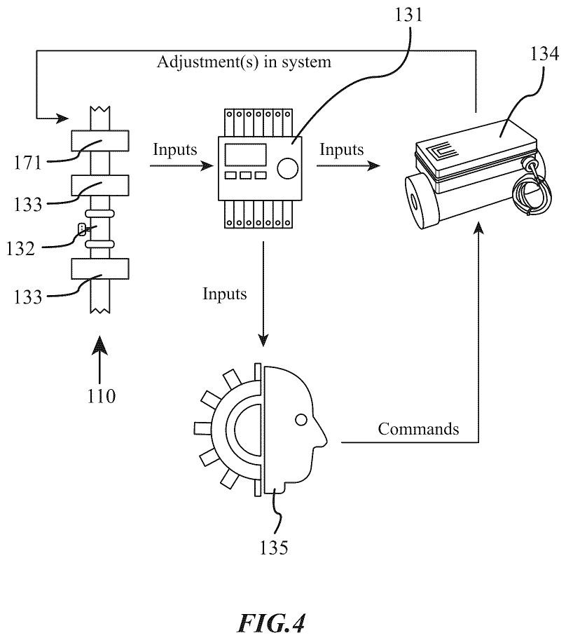

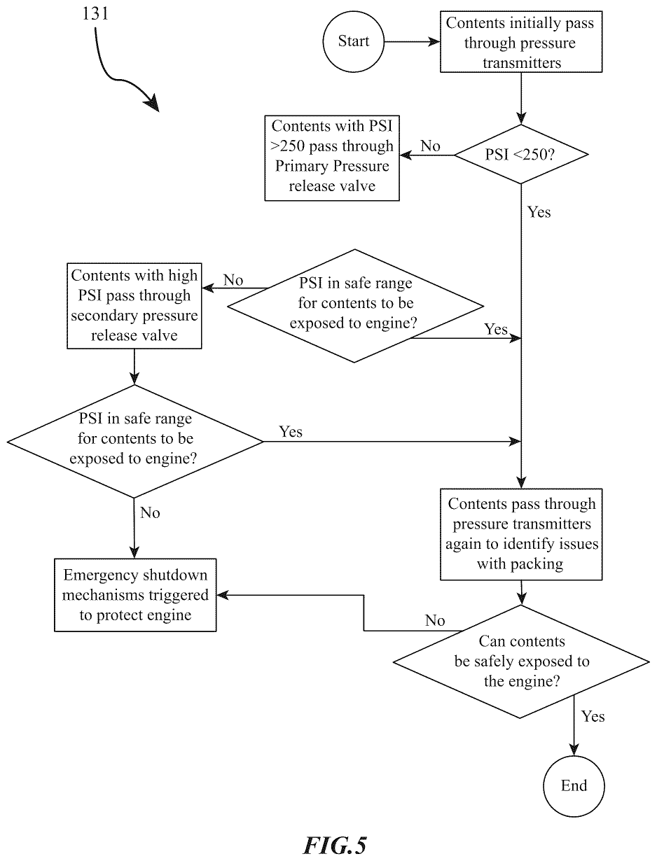

FIG. 4 illustrates the control system architecture in accordance with the preferred embodiment, including the human-machine interface (HMI) 135 that provides operators with real-time monitoring and control capabilities. The safety system components 140, shown in FIG. 5, in accordance with the preferred embodiment incorporates primary pressure relief valve 141 set at 250 psi and secondary pressure relief valve 142 set at 500 psi. Pressure transmitters 143 are strategically placed to detect packing failures, while automatic shutdown mechanisms 144 protect against high-pressure scenarios.

The preferred embodiment integrates various components to effectively capture, process, and utilize fugitive combustible gases, as depicted by FIG. 1. The filtration system 120 incorporates a coalescing filter designed to remove oil and other contaminants from the gas stream. Ball valves are installed on either side of the filtration system 120, enabling isolation of the unit for maintenance without interrupting engine operation. The control system architecture includes a programmable logic controller (PLC) 131 that continuously monitors and adjusts the system based on inputs from various sensors. Flow meters 132 measure the rate of gas flow at critical points in the system, while pressure sensors 133 monitor gas pressures throughout the process. Control valves 134, including the smart control valve, play a critical role in regulating gas flow and pressure, receiving commands from the PLC 131 to adjust their positions. The human-machine interface (HMI) 135 serves as the primary point of interaction between operators and the control system, displaying real-time data on system performance and allowing operators to input commands and adjust system parameters as needed. This integrated control system ensures precise management of the captured gases while maintaining optimal performance and safety parameters.

The safety system components 140 in accordance with the preferred embodiment provide comprehensive protection against system failures and abnormal operating conditions, as illustrated in FIG. 5. The primary pressure relief valve 141 is set to activate at 250 psi, serving as the first line of defense against overpressurization, while the secondary pressure relief valve 142 provides redundant protection with a higher threshold of 500 psi. Pressure transmitters 143 are strategically positioned throughout the system to continuously monitor pressure levels, particularly around compressor cylinder packings where leaks are most likely to occur. These transmitters enable early detection of packing failures by comparing pressure readings from different locations. The automatic shutdown mechanisms 144 are designed to rapidly halt system operation if pressure levels exceed predetermined safety thresholds, protecting both equipment and personnel. When a pressure anomaly is detected by the pressure transmitters 143, the system triggers an alarm to the control panel, allowing the controller to initiate a controlled shutdown sequence through the automatic shutdown mechanisms 144. This integrated safety system ensures multiple layers of protection against potential system failures while maintaining operational integrity.

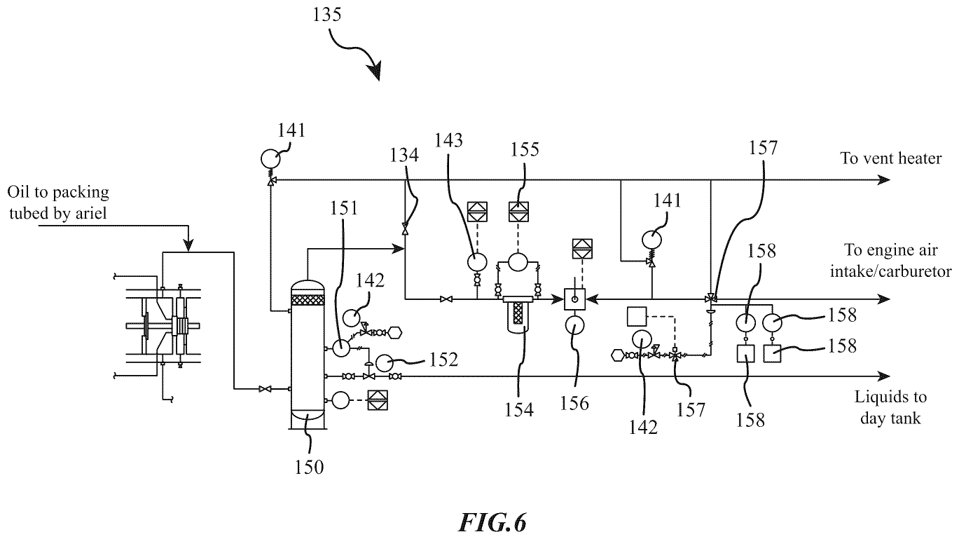

As depicted in FIG. 6, the gas accumulator 150 in accordance with the preferred embodiment collects gas from compressor rod packings and incorporates level control 151 and level control valve 152 to manage liquid separation. Pressure transmitter 153 monitors gas pressure before the stream passes through filter 154. Differential pressure transmitter 155 and flow meter 156 provide crucial monitoring data to the control system. The three-way flow control valve 157 directs gas flow either to the engine or vent, with valve status indicators 158 tracking the flow direction.

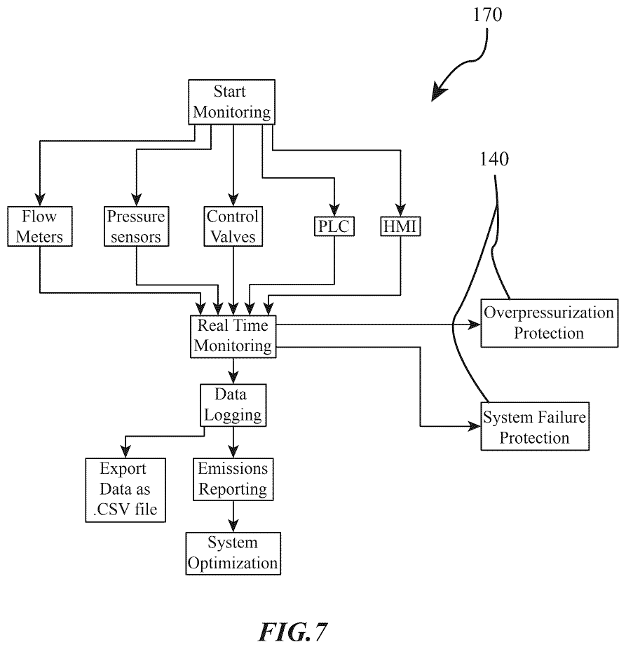

FIG. 7 shows the engine air intake components 160 in accordance with the preferred embodiment, including a dynamic mixing nozzle 161 specifically designed to enhance fuel mixing at the insertion points 162. This configuration ensures optimal mixing of the fugitive gases with the engine's intake air.

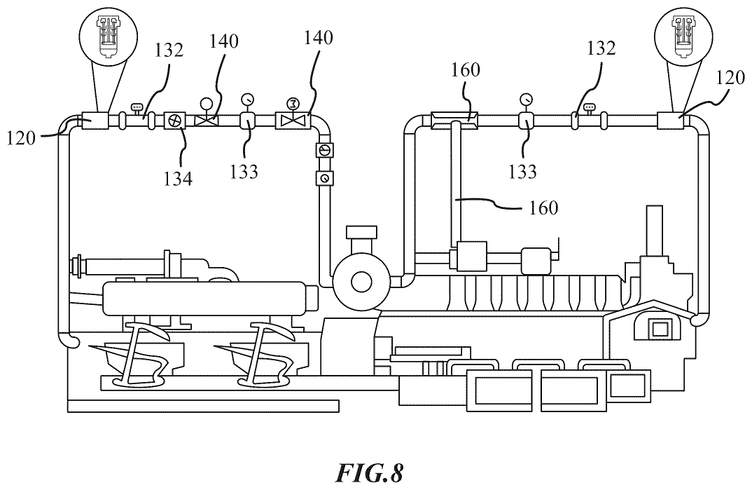

FIG. 8 illustrates aspects of the data logging system 170 in accordance with the preferred embodiment, which incorporates data collection sensors 171 throughout the system, feeding information to the data processing unit 172. This processed data is stored in the data storage system 173, enabling comprehensive emissions tracking and regulatory compliance reporting. The figure demonstrates how these components integrate with existing facility infrastructure.

In typical natural gas production and compression facilities, various components and processes result in the unintentional release of combustible gases into the atmosphere. These fugitive emissions not only represent a loss of valuable fuel but also contribute significantly to environmental concerns due to methane's potent greenhouse effect. The preferred embodiment of the present invention provides a comprehensive solution to mitigate these issues by collecting, processing, and repurposing these otherwise wasted gases.

The system comprises several key components and subsystems working in concert to achieve its objectives. At its core, the invention includes a network of collection points strategically located throughout a facility to capture fugitive gases from multiple sources. These sources may include, but are not limited to, compressor cylinder packings, engine crankcases, instrumentation vents, gas dehydration units, and petroleum liquid storage tanks.

Once collected, the fugitive gases undergo a filtration and processing stage to remove contaminants and ensure the gas is suitable for use as a supplementary fuel source. This processed gas is then carefully introduced into the engine's air intake system, effectively recycling what would otherwise be wasted emissions back into the combustion process.

A sophisticated control and monitoring system forms the backbone of the invention, employing a combination of flow meters, pressure sensors, control valves, and a programmable logic controller (PLC). This system continuously monitors gas flow rates, pressures, and engine parameters to optimize performance and ensure safe operation. A human-machine interface (HMI) provides operators with real-time data and control capabilities, enhancing overall system management and efficiency.

Safety is a paramount consideration in the design of this system. Multiple safeguards are incorporated to protect against potential failures, including overpressurization scenarios and engine malfunctions. These safety features work in tandem with the control system to provide a robust and reliable solution for fugitive gas mitigation.

Furthermore, the invention in the preferred embodiment includes comprehensive data logging and reporting capabilities. This feature not only aids in regulatory compliance by providing accurate emissions data but also enables ongoing system optimization and performance analysis.

The following detailed description will elaborate on each component of the system, their interactions, and the overall operation of the invention. Reference will be made to the accompanying drawings, which provide visual representations of the system's layout, individual components, and key processes.

The preferred embodiment integrates various components to capture, process, and utilize fugitive combustible gases effectively, as depicted by FIG. 1. The system's design incorporates multiple gas collection points, filtration units, control valves, safety devices, and connections to the engine air intake, working in concert to mitigate greenhouse gas emissions and improve fuel efficiency.

Multiple gas collection points are strategically positioned throughout the facility to capture fugitive gases from various sources. These collection points include compressor cylinder packings, engine crankcases, instrumentation vents, gas dehydration units, and petroleum liquid storage tanks. Each collection point is equipped with appropriate ducting and piping to route the captured gases to a central collection system. This comprehensive approach ensures that a wide range of potential emission sources are addressed, maximizing the system's effectiveness in reducing overall greenhouse gas emissions.

Filtration units play a crucial role in processing the collected fugitive gases. The system employs a coalescing filter designed to remove oil and other contaminants from the gas stream. This filtration process is essential to ensure the quality and purity of the gas before it is introduced into the engine air intake. The filter is designed for easy accessibility, allowing for efficient maintenance and replacement. Ball valves are installed on either side of the filter, enabling isolation of the filtration unit without interrupting engine operation. This design feature ensures continuous system operation even during filter maintenance or replacement.

Control valves are integral to managing the flow and pressure of the fugitive gases within the system. A key component is the smart control valve, which maintains the desired pressure of the fugitive gases entering the engine. This valve works in conjunction with a programmable logic controller (PLC) that continuously monitors and adjusts the system based on inputs from various sensors. The control valves enable precise regulation of the gas flow, ensuring optimal mixing with the engine's air intake and maintaining proper fuel-air ratios for efficient combustion.

An exemplary embodiment further comprises a comprehensive vent gas capture control system for managing and monitoring fugitive gases. In an example, an accumulator collects gas from the rod packing distance piece section of the compressor cylinders. Free liquid, which is typically lube oil, from this gas is separated in the accumulator. When a high level is detected by an associated level control device, the liquid is automatically dumped to the lube oil day tank through the opening of the level control valve.

The gas accumulator 150 in accordance with an embodiment serves as a critical component in the vent gas capture control system, collecting gas from the rod packing distance piece section of the compressor cylinders. Free liquid, typically lube oil, is separated within the gas accumulator 150, and when a high level is detected by level control 151, the liquid is automatically dumped to the lube oil day tank through the opening of level control valve 152.

Gas from the accumulator 150 in accordance with an embodiment undergoes pressure measurement via pressure transmitter 153 before being processed through filter 154. The differential pressure transmitter 155 measures and transmits the pressure differential across filter 154 to the local control panel. The volume of collected gas is measured using flow meter 156, with the volume data transmitted to the control system for monitoring and analysis.

The cleaned and metered gas then passes to the three-way flow control valve 157 in accordance with an embodiment, which directs the flow either to the engine for combustion as fuel or to vent. The control system is programmed with specific logic to actuate the three-way flow control valve 157 based on predetermined conditions, including engine RPM and vent gas header pressure reaching desired levels. The status of valve 157 is continuously monitored through valve status indicators 158, with the system calculating the volume of vent gas burned based on the flow rate and valve position. When valve 157 is open, the gas is utilized as fuel, and when closed, the gas is vented.

The system in an embodiment maintains comprehensive monitoring through various input/output components, including an accumulator level switch, a pressure transmitter, a filter, a differential pressure Transmitter, a mass flow meter, a shutdown valve solenoid, and shutdown valve status indicators.

The status of the three-way valve is continuously recorded through shutdown valve status indicators, with the system calculating the volume of vent gas burned based on the flow rate recorded via a mass flow meter and the valve status. When the valve is open, in accordance with an embodiment the gas is utilized as fuel, and when closed, the gas is vented.

This control system integrates with the broader monitoring capabilities of the preferred embodiment, providing real-time data logging and reporting features that enable precise tracking of emissions and system performance. The data collected through this control system contributes to the comprehensive emissions monitoring and regulatory compliance capabilities of the overall system.

Safety devices are incorporated throughout the system to protect against potential failures and abnormal operating conditions. These include primary and secondary pressure relief valves, set at 5 psi and 150 psi respectively, to prevent overpressurization. Pressure transmitters are installed to detect and locate packing failures, providing early warning of potential issues. The system also includes automatic shutdown mechanisms triggered by high-pressure scenarios. These safety features work in tandem to protect the engine and compressor from damage due to system failures or abnormal operating conditions, ensuring the overall reliability and longevity of the system.

The engine air intake components 160 in an embodiment introduce the processed fugitive gases into the engine's combustion system effectively. The cleaned gases are introduced into the engine's air intake through a dynamic mixing nozzle 161 specifically designed to enhance fuel mixing at the insertion point 162. This nozzle ensures proper mixing and distribution of the gases with the incoming air, creating an optimized diluted fuel mixture. The dynamic mixing nozzle 161 is engineered to maintain optimal engine performance while utilizing the captured emissions as a supplementary fuel source. The integration of these components with the engine air intake system 160 is designed to be seamless, allowing for easy retrofitting of existing engine systems without significant modifications.

The collection of fugitive gases from multiple sources within a natural gas production facility is a critical aspect of the system in the context of the invention. This comprehensive approach to gas collection addresses a significant environmental and regulatory challenge faced by the oil and gas industry.

The criticality of this aspect of an embodiment of the invention lies in its ability to capture a wide range of fugitive emissions that would otherwise be released into the atmosphere. By targeting multiple sources such as compressor cylinder packings, engine crankcases, instrumentation vents, gas dehydration units, and petroleum liquid storage tanks, the system maximizes its effectiveness in reducing overall greenhouse gas emissions.

This multi-source collection approach is particularly critical given the stringent regulations surrounding methane emissions in the oil and gas sector. The system directly addresses compliance with regulations such as NSPS OOOO b/c and the Methane Emission Reduction Program (MERP), which place limits on methane emissions and impose costs on excess emissions.

Furthermore, the comprehensive collection of fugitive gases enables the system to transform what would typically be wasted emissions into a valuable fuel source. By capturing these gases and reintroducing them into the engine's air intake, the system not only reduces environmental impact but also improves fuel efficiency and operational performance of the facility.

The criticality of this aspect is further emphasized by its role in enabling accurate monitoring and reporting of emissions. By collecting gases from multiple sources, the system provides a more complete picture of the facility's emissions profile, which is essential for regulatory compliance and for optimizing the facility's environmental performance.

As shown in FIG. 2, the system collects fugitive gases from several sources within a natural gas production facility. These sources include compressor cylinder packings, engine crankcases, instrumentation vents, gas dehydration units, and petroleum liquid storage tanks.

Each collection point is equipped with appropriate ducting and piping to route the fugitive gases to a central collection system, as illustrated in FIG. 2.

Compressor cylinder packings are a significant source of fugitive emissions in natural gas compression facilities. The system includes dedicated vents connected to the compressor cylinder packings to capture these emissions. These vents are designed to handle the high-pressure gas that may escape from the packing seals during normal operation. The captured gas from the compressor cylinder packings is typically rich in methane and other hydrocarbons, making it an ideal candidate for reuse as a supplementary fuel source.

Engine crankcases are another important collection point for fugitive emissions. The system incorporates crankcase vents that are specifically designed to capture the gases that accumulate in the engine crankcase during operation. These gases often contain a mixture of unburned fuel, oil vapors, and combustion byproducts. By capturing and routing these emissions back into the system, the preferred embodiment not only reduces environmental impact but also potentially improves engine efficiency by recirculating unburned fuel.

Instrumentation vents are integrated into the collection system to capture emissions from various control and measurement devices used throughout the facility. These instruments, which may use natural gas as a power source or for pneumatic control, can be a significant source of fugitive emissions. The collection system is designed to accommodate the intermittent and potentially low-volume nature of these emissions, ensuring that even small sources of fugitive gases are captured and utilized.

Gas dehydration units, which are used to remove water vapor from natural gas, can be a substantial source of methane and volatile organic compound (VOC) emissions. The preferred embodiment includes collection points specifically designed to capture emissions from these units. This may involve connecting to existing vent stacks or incorporating new collection piping to route these emissions into the central collection system.

Petroleum liquid storage tanks are also equipped with collection points in the preferred embodiment. These tanks can be a significant source of VOC emissions due to the evaporation of light hydrocarbons from the stored liquids. The collection system is designed to capture these vapors, which not only reduces emissions but also potentially recovers valuable product that would otherwise be lost to the atmosphere.

Each of these collection points is equipped with appropriate ducting and piping to route the fugitive gases to a central collection system. The ducting and piping are designed to handle the specific characteristics of the emissions from each source, such as pressure, temperature, and composition. The system may incorporate flow control devices, such as valves or regulators, to manage the flow of gases from each collection point. This ensures that the central collection system receives a balanced and controlled input of fugitive gases from all sources.

FIG. 3 presents a detailed cross-section of the filtration system. The collected fugitive gases pass through a coalescing filter designed to remove oil and other contaminants. This filtration process is crucial to ensure the quality of the gas before it is introduced into the engine air intake.

The filter is easily accessible for maintenance and replacement, featuring ball valves on either side to allow isolation without interrupting engine operation.

The preferred embodiment incorporates a sophisticated filtration system to process the collected fugitive gases before they are introduced into the engine air intake. At the heart of this system is a coalescing filter designed to remove oil and other contaminants from the gas stream.

The coalescing filter operates by forcing the gas stream through a series of fine mesh or fibrous materials. As the gas passes through these materials, oil droplets and other particulates collide with the filter media and coalesce into larger droplets. These larger droplets then fall to the bottom of the filter housing, where they can be collected and removed. This process effectively separates the liquid contaminants from the gas stream, resulting in a cleaner gas output.

The filtration process is crucial for several reasons. Firstly, it protects the engine from potential damage that could be caused by oil or other contaminants entering the combustion chamber. Secondly, it ensures that the quality of the gas being introduced into the engine air intake is consistent and suitable for efficient combustion. This is particularly important given the variable nature of the fugitive gases being collected from different sources within the facility.

The filter design in the preferred embodiment prioritizes ease of maintenance and system reliability. The filter housing is constructed to allow easy access for inspection, cleaning, and replacement of the filter elements. This design consideration ensures that regular maintenance can be performed quickly and efficiently, minimizing system downtime.

A key feature of the filtration system is the inclusion of ball valves on either side of the filter. These valves serve a critical function in the system's operation and maintenance. When both valves are open, the gas flows through the filter as part of the normal operation. However, when maintenance or replacement of the filter is required, these valves can be closed to isolate the filter from the rest of the system, for example to allow for replacement or maintenance of elements of the system such as the filter.

The ability to isolate the filter without interrupting engine operation is a significant advantage of this design. By closing the ball valves, maintenance personnel can safely remove and service the filter while the engine continues to operate using its primary fuel source. This feature enhances the overall system reliability and reduces potential downtime associated with filter maintenance or replacement.

Furthermore, the ball valve configuration allows for the installation of a bypass line, which can be used to route the gas flow around the filter in case of emergency or during filter change-out procedures. This additional redundancy ensures that the system can continue to operate even in the event of a filter blockage or during maintenance activities.

The filtration system in accordance with an embodiment also incorporates pressure monitoring devices before and after the filter. These devices help operators track the pressure drop across the filter, which is an indicator of filter efficiency and cleanliness. As the filter becomes loaded with contaminants, the pressure drop will increase, signaling the need for maintenance or replacement.

The system in an embodiment further incorporates MKS FTIR (Fourier Transform Infrared) analyzers for comprehensive exhaust monitoring capabilities. These analyzers are specifically configured to measure multiple gas components simultaneously, including NOx, CH4, CO, and O2 concentrations in the engine exhaust.

The MKS FTIR analyzers in accordance with an embodiment are integrated into a compliance testing trailer setup that interfaces with the main system through a dedicated PLC connection. This configuration enables the analyzers to perform detailed emissions tests, typically consisting of three one-hour test runs that generate approximately 180 data points per monitored parameter.

The FTIR analyzer system in accordance with an embodiment works in conjunction with the existing control panel and vent-gas monitoring components to provide comprehensive emissions analysis. The data collected by the FTIR analyzers is synchronized with other system parameters, including engine operational data and vent-gas measurements, enabling detailed correlation between exhaust composition and system performance.

The FTIR analysis system is particularly effective for monitoring variations in exhaust composition that may indicate potential system issues, such as catalyst wear or gasket seal degradation. For example, instability in NOx and CO measurements can serve as early indicators of catalytic converter performance degradation.

The system in accordance with an embodiment may be further enhanced with a stationary emission sensor setup comprising electrochemical sensors for measuring NOx, CO, and O2 concentrations, along with an NDIR (Non-Dispersive Infrared) sensor specifically configured for CH4 detection. These sensors can be permanently mounted to provide ongoing periodic emission analysis, complementing the data collected through other system components.

The electrochemical sensors in accordance with an embodiment utilize selective chemical reactions to detect specific gas components, providing continuous monitoring capabilities for NOx, CO, and O2 levels. The NDIR sensor employs infrared absorption technology particularly suited for methane detection, ensuring accurate measurement of CH4 emissions. This combination of sensor technologies enables comprehensive monitoring of all critical emission components.

The system in accordance with an embodiment implements a dedicated integration protocol between the original system's PLC and the compliance testing trailer. The trailer's PLC interfaces directly with the vent-gas monitor during testing operations, enabling synchronized data collection and control capabilities.

During compliance testing operations, the trailer's PLC establishes communication with the main system's control panel via Modbus or CANbus protocols. This connection enables the synchronized collection of all engine and compressor sensor data, including crankcase pressure, manifold pressure, rpm, and detonation parameters, alongside the emissions data being collected by the trailer's analyzers.

The integration system in an exemplary embodiment is designed to collect approximately 180 data points per parameter during typical three-hour test runs. The synchronized data collection ensures that emissions measurements from the trailer's MKS FTIR analyzers are precisely time-aligned with the operational data from the main system's sensors and control panel.

The PLC integration in accordance with an embodiment enables real-time correlation between the vent-gas monitoring system's measurements and the compliance trailer's emissions analysis. All collected data—including vent-gas measurements, exhaust emissions, operational parameters, and process data—is synchronized and uploaded to the cloud platform for comprehensive analysis.

When the compliance testing trailer is deployed in accordance with an embodiment, its PLC automatically synchronizes with the main system's timing protocols to ensure precise temporal alignment of all collected data. This synchronization is crucial for accurate correlation between operational events and corresponding emissions measurements, enabling detailed analysis of system performance and potential issues.

The SCADA system integration in accordance with an embodiment utilizes standardized industrial protocols to enable seamless data transmission between the vent-gas monitoring system and facility control infrastructure. The system supports both cellular/Wi-Fi data transmission capabilities and direct SCADA system integration for periodic data uploads.

In an embodiment of the invention, the SCADA integration protocol enables configurable data transmission intervals, allowing facilities to set specific schedules for data upload and analysis. For example, in a preferred embodiment, operators can configure the system to transmit data hourly, daily, or at custom intervals depending on operational requirements and network bandwidth considerations. When integrated with an existing SCADA system, the protocol establishes automated data collection routines that capture and transmit multiple categories of operational information. In an example of the invention, these data categories include engine and compressor operational data comprising parameters such as RPM, temperatures, pressures, and vibration measurements; comprehensive emissions sensor readings from both standard sensors and specialized analyzers; detailed vent-gas flow measurements and pressure readings from multiple collection points; and system status indicators and alarm conditions that provide real-time operational awareness. For facilities with existing SCADA infrastructure, an embodiment of the invention implements a direct integration pathway utilizing industry-standard protocols such as Modbus, OPC-UA, or DNP3 to enable seamless communication between systems. This integration pathway, in a preferred embodiment of the invention, facilitates automated data collection from all system sensors without requiring manual intervention, enables scheduled transmission of collected data at configurable intervals determined by facility requirements, provides comprehensive integration with facility-wide monitoring and control systems for unified operations management, and establishes secure remote access to system data and control parameters through the facility's existing network infrastructure. The SCADA integration capabilities in an embodiment of the invention transform the disconnected data streams into a cohesive operational intelligence platform that enhances both regulatory compliance documentation and predictive maintenance capabilities.

The data transmission protocol incorporates error checking and validation to ensure data integrity during transmission. When operating in cellular/Wi-Fi mode, the system employs secure transmission protocols to protect sensitive operational data. For SCADA integration, the system supports standard industrial protocols to maintain compatibility with existing facility infrastructure.

The periodic data transmission capabilities in accordance with an embodiment enable long-term trend analysis and system optimization. By collecting and transmitting data at regular intervals, the system builds a comprehensive operational history that can be analyzed for performance optimization and predictive maintenance purposes. This data transmission framework supports both immediate operational monitoring and long-term analysis needs.

The system incorporates SCADA (Supervisory Control and Data Acquisition) integration capabilities in an embodiment, allowing for automated data transmission and analysis at configurable intervals. When connected to a facility's SCADA system, the emission sensor data can be transmitted periodically (e.g., hourly or daily) for systematic analysis and trending.

The SCADA integration enables automated data collection and transmission from both the stationary sensors and the main system components. This integration can be configured to transmit data at user-defined intervals, facilitating long-term monitoring and analysis of system performance. The SCADA system's data transmission capabilities allow for remote monitoring and analysis of emissions data, enhancing the overall effectiveness of the emissions monitoring and control system.

The combination of stationary sensors and SCADA integration provides extended monitoring capabilities, particularly valuable when initial warnings emerge from the system. For example, if the system detects unstable O2 readings or irregular crankcase pressure, the stationary sensors can provide continuous monitoring data through the SCADA system for enhanced analysis and early detection of potential equipment issues.

The AI model in the context of an exemplary embodiment employs pattern recognition to analyze multiple data streams simultaneously for predictive failure analysis. The system in an embodiment specifically examines correlations between engine sensor data, compressor operational parameters, and emissions data to identify potential equipment failures before they occur.

In an embodiment of the invention, the AI analysis focuses on several key pattern combinations that indicate specific types of potential failures. These pattern combinations represent technical implementations that transform disconnected sensor data into actionable maintenance insights.

In a preferred embodiment of the invention, Engine Analysis functionality monitors correlations between O2 fluctuations and manifold pressure instability. When these parameters show coordinated irregularities, they may indicate potential engine problems such as valve train wear or developing piston issues. The system implements this correlation analysis by first normalizing oxygen sensor readings and manifold pressure data against baseline operational parameters, then applying time-series correlation algorithms to identify synchronous deviations that exceed predefined thresholds while filtering normal operational variations. The coordinated pattern detection employs sliding window analysis to detect subtle changes over time that would be imperceptible through traditional threshold-based monitoring systems.

In an example of the invention, Compressor Analysis capabilities track relationships between packing vent flow measurements and pressure readings. A pattern of increasing packing vent flow combined with rising pressure measurements can signal impending packing failure. This analysis implements regression techniques to establish baseline correlation models between these parameters during normal operation, then calculates deviation scores as operational conditions change. The system applies statistical significance testing to distinguish random fluctuations from systematic pattern changes, enabling early detection of developing issues before conventional monitoring systems would trigger alarms.

In an embodiment of the invention, Catalytic Converter Analysis examines patterns of NOx and CO instability in combination. Concurrent instability in these parameters often indicates early signs of catalyst wear or developing gasket seal issues. The analysis implementation converts raw emissions data into normalized ratio values and applies frequency domain transformation to identify characteristic oscillation patterns associated with specific failure modes. This approach enables the system to distinguish between normal operational variations and the distinctive signature patterns that precede catalyst degradation by analyzing the temporal relationship between NOx and CO fluctuations.

In a preferred embodiment, Process Analysis monitors correlations between temperature variations and pressure stability. The combination of rising temperatures with pressure instability patterns can indicate potential compressor valve wear. The system implements this by applying multivariate analysis to temperature and pressure time-series data, identifying leading indicators where temperature changes precede pressure instability by specific time intervals. This predictive relationship is continuously refined through machine learning algorithms that adjust correlation weights based on confirmed valve inspection results, improving detection accuracy over time.

The AI model processes approximately 180 data points per parameter during each analysis cycle in the context of an exemplary embodiment, enabling detailed pattern recognition across multiple operational parameters. This comprehensive analysis allows the system to generate specific probability-based predictions, such as identifying a “65% chance of engine failure in 72 hours” based on the detected pattern combinations.

The system in the context of an exemplary embodiment employs a Long Short-Term Memory (LSTM) model hosted on a cloud platform to analyze the combined sensor and emissions data for failure signatures. This AI architecture enables the system to learn and improve its prediction accuracy over time by analyzing historical pattern data alongside actual equipment failure incidents.

When the AI system detects concerning pattern combinations, such as unstable O2 readings or irregular crankcase pressure, it can trigger enhanced monitoring protocols. These may include activation of the stationary sensor setup for extended data collection and more refined AI analysis of the developing patterns.

The system incorporates enhanced failure prediction capabilities that generate specific probability assessments based on comprehensive data analysis. The AI model processes data from multiple sensors and emissions monitoring systems to calculate precise probability estimates for potential equipment failures.

The failure prediction system generates specific quantified assessments in accordance with an embodiment, such as predicting a “65% chance of engine failure in 72 hours” based on analyzed data patterns. These probability assessments are derived from the analysis of multiple correlated parameters. The system employs machine learning algorithms to continuously monitor and evaluate critical operational data streams simultaneously, applying weighted significance to various indicators based on their historical correlation with confirmed failure events. The predictive calculations incorporate both real-time measurements and trending data, enabling the generation of time-based probability forecasts with specific confidence intervals.

Engine Component Analysis in the context of an exemplary embodiment involves the continuous monitoring of multiple interrelated parameters to detect potential failure conditions before they manifest as operational disruptions. The system examines correlations between O2 fluctuations and manifold pressure instability by applying time-series analysis to identify synchronous variations that exceed normal operational thresholds. When oxygen readings in the exhaust stream demonstrate cyclical deviations that correspond temporally with manifold pressure variations, the system identifies this signature as indicative of developing combustion irregularities. This correlation analysis utilizes pattern recognition algorithms that filter normal operational variations from potentially problematic synchronous fluctuations that typically precede valve train or fuel delivery system issues.

Valve train wear indicator analysis in the context of an exemplary embodiment involves real-time acoustic signature examination overlaid with timing pattern recognition. The system captures high-frequency vibration profiles during valve opening and closing events, comparing these against baseline performance models. As valve components experience progressive wear, characteristic changes in the acoustic signature emerge, particularly in the 2-5 kHz frequency range where valve seating events produce identifiable patterns. The system correlates these acoustic signatures with exhaust composition variations to improve prediction accuracy, as inefficient valve operation typically manifests in corresponding changes to combustion efficiency metrics.

Piston performance metrics monitoring in the context of an exemplary embodiment encompasses several dimensions of engine operation. The system analyzes combustion pressure curves, examining peak pressure timing, rate of pressure rise, and pressure decay characteristics. These measurements are supplemented by crankcase pressure monitoring, which detects minute variations in blow-by gas volume that can indicate ring wear or cylinder wall scoring. Temperature gradient analysis across the cylinder further enhances the system's ability to identify developing piston issues, as uneven heat distribution often precedes mechanical failure. The AI model correlates these parameters with operational variables such as load and RPM to normalize readings across different operating conditions.

Compressor System Analysis in the context of an exemplary embodiment leverages multiple data streams to predict potential failures with high accuracy. The relationship between packing vent flow and pressure measurements provides critical insights into packing integrity. The system establishes baseline correlations between these parameters during normal operation, then continuously calculates deviation percentages that indicate progressive degradation. When packing begins to wear, characteristic changes in the relationship between flow and pressure emerge, with flow rates typically increasing disproportionately to pressure as sealing effectiveness diminishes. The system applies regression analysis to these trends, enabling prediction of remaining useful life based on degradation rates.

Early detection of potential packing failures in the context of an exemplary embodiment is accomplished through multi-parameter analysis that extends beyond simple threshold monitoring. The system examines temperature gradients across packing sections, vibration signature analysis in specific frequency ranges associated with seal interface dynamics, and chemical composition analysis of the vent gas stream. Microscopic quantities of lubrication contaminants in the vent gas often provide the earliest indication of developing packing issues, appearing well before volumetric flow increases become apparent. The AI model weighs these indicators according to their statistical correlation with historical failure data to generate specific probability assessments.

Pressure stability pattern analysis in the context of an exemplary embodiment employs high-resolution sampling to detect subtle indicators of developing compressor issues. The system captures pressure data at 5-10 samples per second, applying both time-domain and frequency-domain analysis techniques to identify characteristic signatures of valve wear, ring damage, or piston issues. The AI model can distinguish between random fluctuations and the systematic patterns indicative of specific failure modes by applying pattern recognition algorithms that have been trained on extensive historical failure data. The progression rate of these patterns enables the system to quantify probability timeframes with significant precision.

Catalytic Converter Performance monitoring in the context of an exemplary embodiment focuses on complex relationships between multiple exhaust components. NOx and CO stability measurements provide direct insight into catalyst efficiency. The system establishes normal fluctuation ranges during optimal operation, then calculates statistical deviation scores when emissions patterns change. These deviations are analyzed in the context of operational parameters such as load, temperature, and air-fuel ratio to isolate catalyst performance from other variables. When the NOx/CO ratio variations exceed established thresholds while showing characteristic patterns, the AI model generates specific probability assessments for catalyst degradation or failure.

Catalyst wear pattern analysis in the context of an exemplary embodiment involves examining the relationship between catalyst bed temperature profiles, conversion efficiency across different exhaust components, and back-pressure measurements. As catalysts degrade, distinctive temperature distribution patterns emerge, with hotspots developing in areas experiencing accelerated degradation. The system tracks conversion efficiency across multiple exhaust constituents simultaneously, as different catalyst materials and contaminants produce characteristic degradation signatures affecting specific components preferentially. These parameters are processed through the AI model to quantify remaining catalyst life expectancy with specific probability distributions.

Gasket seal integrity indicators in the context of an exemplary embodiment are monitored through several complementary approaches. The system performs pressure differential analysis across sealed interfaces, detecting minute leakage rates before they become significant. Acoustic signature detection identifies the characteristic high-frequency components produced by gas escaping through developing seal failures. Trace gas measurements in the exhaust stream can further identify specific gasket materials when they begin to deteriorate. The AI model correlates these parameters with thermal cycling data and operational history to predict seal failure probability, accounting for the known deterioration rates of different gasket materials under various operating conditions.

By integrating these multiple analysis streams, the system generates comprehensive failure predictions with specific probability assessments. The quantified nature of these assessments enables maintenance teams to prioritize interventions based on risk profiles rather than arbitrary maintenance schedules, significantly reducing both unplanned downtime and unnecessary preventive maintenance. The system's continuous learning capability further refines these predictions over time, incorporating outcomes from previous assessments to improve future accuracy.

The probability assessment system utilizes machine learning algorithms to continuously refine its prediction accuracy. The system learns from historical failure data and actual equipment performance outcomes to improve the precision of its probability calculations. This learning capability enables the system to generate increasingly accurate predictions over time.

When the system detects concerning patterns, it initiates a more detailed analysis phase that combines multiple monitoring approaches and advanced analytical methods. This phase begins with extending the monitoring period beyond standard intervals, typically transitioning from hourly snapshots to continuous data collection using the stationary electrochemical and NDIR sensors. The system automatically adjusts sampling rates from standard operational frequency (typically once per minute) to high-resolution monitoring (typically 5-10 data points per second) to capture transient events and subtle parameter variations that might otherwise be missed.

Simultaneously, the system integrates supplementary data points from the compliance testing trailer's MKS FTIR analyzers, which provide high-precision measurements of exhaust gas composition including NOx, CH4, CO, and O2 levels at approximately 180 data points per monitored parameter during a typical three-hour test run. This integration occurs through the dedicated PLC connection that synchronizes timestamps between the main system and the trailer's analyzers, ensuring temporal alignment of all collected data streams.

The enhanced analysis process employs a multi-layered correlation approach wherein the system examines relationships between multiple sensor inputs using both time-domain and frequency-domain analysis techniques. For example, when the initial analysis identifies potential compressor issues, the system specifically examines correlations between packing vent flow measurements, crankcase pressure readings, manifold pressure stability, and emissions data patterns to validate the initial probability assessment. This cross-parameter validation substantially reduces false positives by requiring confirmation across multiple independent measurement domains.

Based on this expanded dataset and cross-correlation analysis, the system continuously recalculates failure probability estimates at configurable intervals (typically every 15 minutes during enhanced monitoring). These probability adjustments incorporate newly acquired data using weighted averaging algorithms that progressively increase the influence of recent measurements while maintaining historical context. For critical parameters approaching threshold values, the system may further increase monitoring resolution and adjust its probability calculations to reflect accelerating degradation patterns, enabling more precise prediction of potential failure timeframes.

This analytical approach enables the system in an embodiment to not only validate initial concerns but also to track the progression of potential issues with increasing precision as additional data becomes available, ultimately providing operations personnel with actionable insights regarding both the nature and timing of required maintenance interventions.

The probability assessments are continuously updated based on incoming data from all system components, including the vent-gas monitor, compliance testing trailer, and any installed stationary sensors. This comprehensive data integration enables the system to provide dynamic, real-time updates to failure probability estimates as conditions change.

The system supports integration with portable electrochemical analyzers, enabling flexible deployment of emissions monitoring capabilities. These portable analyzers can be utilized during annual emission tests in conjunction with the FTIR analyzer system, providing complementary data collection capabilities.

When facilities equipped with the vent-gas monitor require extended analysis periods, a temporary electrochemical sensor setup can be deployed. This portable configuration provides the same analytical capabilities as permanently mounted sensors, allowing for detailed emissions monitoring over extended timeframes while maintaining full integration with the system's AI analysis capabilities.

The system includes a software interface specifically designed to enable facility technicians to perform AI analysis using portable analyzers. When purchased alongside the vent-gas monitor, these portable units come equipped with dedicated software that establishes communication with the vent-gas monitoring system. This integration enables technicians to conduct independent AI-driven analysis using their portable equipment.

The portable analyzer software system maintains seamless data integration with the vent-gas monitor, enabling the same comprehensive AI analysis capabilities available through permanent installations. This flexibility allows facilities to conduct detailed emissions analysis and failure prediction assessments using either permanent or portable monitoring configurations, while maintaining consistent analytical capabilities.

The control system in the preferred embodiment serves as the central intelligence unit, orchestrating the various components to ensure optimal performance and safety. FIG. 4 depicts this control system, which comprises several key elements working in concert in accordance with an embodiment.

At the heart of the control system is a programmable logic controller (PLC). The PLC is a digital computer designed for industrial applications, capable of executing complex control algorithms and processing inputs from multiple sources simultaneously. In this embodiment, the PLC is programmed to continuously monitor and adjust the system based on real-time data from various sensors throughout the installation.

Flow meters are integrated into the control system in an embodiment to measure the rate of gas flow at critical points in the system. These meters provide essential data on the volume of fugitive gases being captured and processed, as well as the flow rates into the engine air intake. This information is crucial for maintaining proper fuel-air ratios and optimizing engine performance.

Pressure sensors are strategically placed throughout the system to monitor gas pressures at various stages of the process. These sensors provide vital data on system operation, helping to detect potential issues such as blockages or leaks, and ensuring that gas pressures remain within safe operating limits.

Control valves, including the smart control valve in accordance with an embodiment, play a critical role in regulating gas flow and pressure. These valves receive commands from the PLC to adjust their positions, thereby controlling the flow of gases through the system. The smart control valve, in particular, is designed to maintain the desired pressure of the fugitive gases as they enter the engine, ensuring consistent and efficient fuel delivery.

The human-machine interface (HMI) serves as the primary point of interaction between operators and the control system. This interface displays real-time data on system performance, including engine speed, fuel flow rates, methane concentrations, and pressure readings. The HMI also allows operators to input commands and adjust system parameters as needed.

The PLC's continuous monitoring and adjustment capabilities are central to the system's effectiveness. It receives inputs from all sensors in real-time, processes this data according to its programmed logic, and sends appropriate control signals to the various actuators in the system, such as control valves. This constant feedback loop allows the system to respond quickly to changing conditions, maintaining optimal performance and safety.

The smart control valve, which in an embodiment comprises a configuration of a smart control valve as commonly known to be manufactured by Continental Controls is a key component in maintaining the desired pressure of the fugitive gases entering the engine. This valve incorporates its own pressure transmitter and control logic, allowing it to respond rapidly to pressure fluctuations. The valve maintains the pressure that is specified by the PLC, adjusting its position as needed to compensate for variations in gas flow or engine demand. This precise pressure control is crucial for ensuring stable and efficient engine operation when using the captured fugitive gases as a supplementary fuel source.

The preferred embodiment incorporates a comprehensive safety system to protect the engine and compressor from potential damage due to system failures or abnormal operating conditions. FIG. 5 illustrates these safety systems in accordance with an embodiment.

Primary and secondary pressure relief valves are key components of the safety system. The primary relief valve is set to activate at 5 psi, while the secondary valve is set at a higher threshold of 150 psi in accordance with an embodiment. This dual-valve configuration provides redundancy and ensures that the system remains protected even if one valve fails to operate. The primary valve acts as the first line of defense against overpressurization, relieving excess pressure before it reaches critical levels. If the primary valve fails or is unable to relieve pressure quickly enough, the secondary valve serves as a backup, preventing catastrophic failure.

Pressure transmitters are strategically placed throughout the system to detect and locate packing failures. These transmitters continuously monitor pressure at various points, particularly around compressor cylinder packings where leaks are most likely to occur. By comparing pressure readings from different locations, the control system can identify anomalies that may indicate a packing failure. This early detection capability allows for prompt intervention, preventing minor issues from escalating into major failures.

The system also incorporates automatic shutdown mechanisms triggered by high-pressure scenarios. These mechanisms are designed to rapidly halt system operation if pressure levels exceed predetermined safety thresholds. The shutdown process may involve closing valves to isolate high-pressure areas, stopping the compressor, and cutting fuel supply to the engine. This rapid response capability is crucial for preventing equipment damage and ensuring personnel safety in the event of a sudden pressure spike.

These safety features work in concert to protect the engine and compressor from potential damage. By continuously monitoring system pressures and providing multiple layers of protection against overpressurization, the safety system can effectively mitigate risks associated with system failures or abnormal operating conditions.

In the event of a compressor packing failure, for example, the safety system would respond in a coordinated manner. The pressure transmitters would detect the abnormal pressure increase, triggering an alarm to the control panel. This would allow the controller to initiate a controlled shutdown of the unit. Simultaneously, if the pressure continues to rise, the primary relief valve would activate to relieve the excess pressure. In the unlikely event that this is insufficient, the secondary relief valve provides an additional safeguard.

The automatic shutdown mechanisms are particularly important in scenarios where rapid intervention is necessary. For instance, if a sudden blockage in the system causes a rapid pressure increase, the shutdown system can react faster than human operators, potentially preventing catastrophic equipment failure.

By integrating these safety features, the preferred embodiment ensures robust protection against a wide range of potential failure modes. This comprehensive approach not only safeguards the expensive engine and compressor equipment but also contributes to the overall safety and reliability of the natural gas production facility.

An embodiment of the invention comprises a user interface, optionally comprising a human-machine interface. The human-machine interface (HMI) depicted in FIG. 6 serves as the primary point of interaction between operators and the control system, providing a comprehensive and user-friendly interface for monitoring and controlling the Engine and Compressor Vent Gas Mitigation System.

The HMI displays real-time data on system performance through a series of digital readouts, graphs, and visual indicators. Key performance metrics shown on the interface in accordance with an embodiment include one or more of the following:
Engine speed: A digital readout displays the current RPM of the engine, allowing operators to monitor its operational status and ensure it remains within optimal parameters.
Fuel flow rates: The interface presents both the flow rate of the primary natural gas fuel and the supplementary fugitive gas fuel. This information is crucial for operators to understand the proportion of fugitive gases being utilized and the overall fuel consumption of the engine.
Methane concentrations: The HMI provides readings of methane concentrations at various points in the system, including the compressor vent, engine crankcase vent, and engine exhaust. These measurements are essential for monitoring the effectiveness of the fugitive gas capture system and ensuring that methane emissions are being properly managed.
Pressure readings: The interface displays pressure data from multiple sensors throughout the system, including the compressor vent pressure, crankcase pressure, and engine air inlet pressure. These readings help operators identify potential issues such as blockages or leaks in the system.
Calculated greenhouse gas metrics: The HMI processes data from various sensors to provide real-time calculations of greenhouse gas emissions. This may include metrics such as CO2 equivalent emissions per hour, allowing operators to track the system's environmental impact continuously.

The user interface in accordance with the preferred embodiment is designed with a user-friendly layout, utilizing color-coded indicators and graphical representations to make data interpretation intuitive and efficient. For example, pressure readings might be displayed on gauge-style indicators, while fuel flow rates could be represented by dynamic bar graphs.

In addition to displaying data, the HMI allows operators to input commands and adjust system parameters. This may include features such as one or more of the following:
Control valve adjustment: Operators can fine-tune the position of control valves to optimize the flow of fugitive gases into the engine air intake.
Alarm settings: The interface allows for the configuration of alarm thresholds for various parameters, ensuring that operators are promptly alerted to any abnormal conditions.
Data logging controls: Operators can initiate or adjust data logging settings, enabling comprehensive record-keeping for regulatory compliance and performance analysis.

By providing this real-time information and control capabilities, the HMI empowers

operators to make informed decisions that optimize system performance and reduce emissions. For instance, if the interface indicates an increase in methane concentration in the engine exhaust, operators can adjust the air-fuel ratio to ensure more complete combustion. Similarly, if pressure readings suggest a potential blockage in the filtration system, operators can initiate maintenance procedures before the issue affects overall system performance.

The HMI also plays a crucial role in emissions reduction by allowing operators to monitor and fine-tune the system's performance continuously. By providing real-time greenhouse gas metrics, the interface enables operators to assess the immediate impact of their control decisions on emissions levels. This immediate feedback loop facilitates a proactive approach to emissions management, allowing for rapid adjustments to maintain optimal environmental performance.

Furthermore, the data presented on the HMI can be used for long-term performance analysis and optimization. The system's ability to log data over time allows for the identification of trends and patterns in engine performance and emissions levels. This historical data can inform maintenance schedules, guide system upgrades, and support continuous improvement efforts to further reduce emissions and enhance operational efficiency.

The data logging system 170 in accordance with an embodiment is designed to capture and record information at a high frequency, logging data on a per-second basis through data collection sensors 171 positioned throughout the system. This granular approach to data collection provides an exceptionally detailed record of all process and operating parameters within the system. By capturing data at such frequent intervals through the sensors 171, the system can accurately track rapid changes in operating conditions, allowing for precise analysis of system performance and emissions output.

The data processing unit 172 in accordance with an embodiment handles various types of data, including engine speed, fuel flow rates (both for the primary natural gas fuel and the supplementary fugitive gas), methane concentrations at various points in the system, pressure readings from multiple sensors, and calculated greenhouse gas metrics. This comprehensive data set processed by unit 172 provides a complete picture of the system's operation and environmental impact.

One of the key features of the data storage system 173 in accordance with an embodiment is its ability to export data in a widely compatible format. The system generates a . csv (comma-separated values) file for each calendar day, containing all the logged data from sensors 171. This file format is chosen for its versatility and ease of use with various analysis tools and software packages. The daily export feature allows for convenient segmentation of data, facilitating both short-term operational analysis and long-term trend identification.

The exportable nature of the data from storage system 173 in accordance with an embodiment is particularly valuable for regulatory compliance purposes. Many environmental regulations require detailed reporting of emissions and operational data. By providing easily accessible and comprehensive data files, the system simplifies the process of generating required reports for regulatory bodies. This feature can significantly reduce the administrative burden associated with compliance reporting.

For performance optimization, the detailed data logs stored in system 173 in accordance with an embodiment enable engineers and operators to conduct in-depth analysis of system behavior. By examining trends and correlations in the data collected by sensors 171 and processed by unit 172, they can identify opportunities for improving efficiency, reducing emissions, or enhancing overall system performance. The high-frequency data collection allows for the detection of subtle patterns or brief anomalies that might be missed with less frequent logging.

In terms of emissions tracking, the system's ability to log calculated greenhouse gas metrics through sensors 171 in accordance with an embodiment on a per-second basis provides an unprecedented level of detail in emissions monitoring. This granular data allows for precise quantification of the system's environmental impact and can be used to demonstrate the effectiveness of the fugitive gas capture and utilization process. It also enables rapid detection of any unexpected increases in emissions, allowing for prompt corrective action.

The combination of comprehensive data collection through sensors 171, processing through unit 172, and storage in system 173 makes this system in accordance with an embodiment a powerful tool for ongoing system management and improvement. It provides the foundation for data-driven decision-making, enabling operators to fine-tune system parameters based on empirical evidence rather than estimation or intuition.

FIG. 8 provides a detailed illustration of how an embodiment of the system in accordance with an embodiment integrates with an existing engine and compressor setup in a typical industrial environment. This diagram serves to highlight the system's adaptability and ease of installation across various facility configurations.

The diagram demonstrates how the system can be retrofitted to existing equipment without requiring significant modifications to the core engine and compressor infrastructure. It shows the strategic placement of collection points for fugitive gases, including connections to compressor cylinder packings, engine crankcases, instrumentation vents, and other potential emission sources.

The integration diagram illustrates the routing of collected fugitive gases through the filtration system, which is positioned to allow easy access for maintenance while minimizing disruption to existing operations. The placement of control valves and safety devices is shown in relation to the engine and compressor, emphasizing how these components can be incorporated without interfering with normal equipment function.

FIG. 8 also depicts the connection points where the processed fugitive gases are introduced into the engine's air intake system. This visualization helps to demonstrate how the system can utilize existing air intake infrastructure with minimal modifications, further emphasizing its adaptability.

The diagram includes representations of the control system components, such as the PLC and HMI, showing how these can be positioned for optimal operator access and system monitoring. This layout consideration underscores the system's focus on user-friendly operation and seamless integration into existing control environments.

By providing a visual representation of the system's integration, FIG. 8 illustrates the flexibility of the design to accommodate various facility layouts and equipment configurations. This adaptability is crucial for the system's practical implementation across a wide range of natural gas production facilities, each with its unique spatial constraints and operational requirements.

The preferred embodiment addresses several key regulatory requirements related to methane emissions and greenhouse gas reduction in the oil and gas industry. Specifically, the system is designed to comply with regulations such as NSPS OOOO b/c and the Methane Emission Reduction Program (MERP), which place limits on methane emissions and impose costs on excess emissions.

The system's comprehensive approach to capturing fugitive gases from multiple sources within a natural gas production facility directly addresses the regulatory caps on venting natural gas. By collecting emissions from compressor cylinder packings, engine crankcases, instrumentation vents, gas dehydration units, and petroleum liquid storage tanks, the system helps facilities stay within the allowed venting limits, such as the two standard cubic feet per minute per compressor throw.

The data logging and reporting capabilities of the system play a crucial role in regulatory compliance. The system logs data on a per-second basis, providing detailed information on all process and operating parameters. This granular data collection allows for precise quantification of emissions, which is essential for demonstrating compliance with regulatory limits. The ability to easily export this data as daily . csv files facilitates efficient reporting to regulatory bodies, reducing the administrative burden associated with compliance reporting.

Furthermore, the system's real-time monitoring and control capabilities enable operators to quickly identify and address any deviations from regulatory limits. The human-machine interface (HMI) provides continuous updates on methane concentrations and calculated greenhouse gas metrics, allowing for immediate corrective action if emissions approach regulatory thresholds.

The system's ability to capture and utilize fugitive gases as a supplementary fuel source not only reduces emissions but also aligns with regulatory goals of improving energy efficiency and reducing waste. This dual benefit of emissions reduction and energy recovery demonstrates a proactive approach to meeting and exceeding regulatory requirements.

By providing accurate, real-time measurement of vented emissions, the system addresses a key regulatory concern regarding the quantification and reporting of greenhouse gas emissions. This capability is particularly relevant in the context of regulations like the Waste Emissions Charge (WEC) under the Inflation Reduction Act, which imposes fees on excess methane emissions.

The Waste Emissions Charge (WEC) under the Inflation Reduction Act represents a significant regulatory measure aimed at reducing methane emissions in the oil and gas industry. This charge imposes escalating fees on excess methane emissions, creating a strong financial incentive for companies to minimize their emissions. Specifically, the WEC is set to increase from $900 per metric ton of methane in 2024 to $1,200 per metric ton in 2025, and further to $1,500 per metric ton in 2026.

The system's capability to accurately measure and report fugitive gas emissions is crucial in this regulatory context. By providing precise, real-time data on methane emissions, the system enables operators to closely monitor their emission levels and take proactive measures to stay below the thresholds that would trigger these fees. This not only helps companies avoid significant financial penalties but also aligns their operations with the broader policy goal of reducing greenhouse gas emissions. Furthermore, the detailed data logging and reporting features of the system can provide the documentation necessary to demonstrate compliance with WEC regulations, potentially saving companies substantial amounts in avoided fees while contributing to national emission reduction targets.

The preferred embodiment offers several key benefits that make it a comprehensive solution for capturing, processing, and utilizing fugitive combustible gases in natural gas production facilities. These benefits collectively contribute to improved environmental performance, operational efficiency, and regulatory compliance.

Firstly, the system significantly reduces greenhouse gas emissions by capturing and utilizing fugitive gases that would otherwise be released into the atmosphere. This not only mitigates the environmental impact of natural gas production but also aligns with increasingly stringent regulations on methane emissions. The system's ability to convert these captured gases into a supplementary fuel source for the engine represents a dual benefit of emissions reduction and energy efficiency.

Secondly, the system's adaptability and ease of integration with existing equipment make it a practical solution for a wide range of facility configurations. As demonstrated in FIG. 8, the system can be retrofitted to existing engine and compressor setups with minimal disruption to ongoing operations. This flexibility ensures that the benefits of fugitive gas capture can be realized across diverse industrial environments.

Thirdly, the comprehensive control and monitoring capabilities provided by the PLC and HMI allow for precise management of the system. Real-time data on system performance, including engine speed, fuel flow rates, and methane concentrations, enables operators to make informed decisions that optimize performance and further reduce emissions. This level of control contributes to improved overall operational efficiency.

Fourthly, the robust safety features incorporated into the system, including pressure relief valves and automatic shutdown mechanisms, ensure protection against potential equipment damage due to system failures or abnormal operating conditions. This enhances the reliability and longevity of both the fugitive gas capture system and the existing engine and compressor equipment.

Finally, the detailed data logging and reporting capabilities of the system provide significant advantages for regulatory compliance and performance optimization. The ability to generate comprehensive, easily exportable data files facilitates efficient reporting to regulatory bodies and enables in-depth analysis for continuous system improvement.

The preferred embodiment of the invention represents a holistic approach to addressing the challenges of fugitive gas emissions in natural gas production facilities. By combining effective emissions capture, energy efficiency improvements, advanced control and monitoring capabilities, robust safety features, and comprehensive data management, the system offers a solution that is both environmentally responsible and operationally beneficial.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A system for predictive analysis of natural gas equipment, comprising:
a) a vent-gas monitoring system configured to:
collect fugitive gases from multiple sources;
measure flow and pressure from compressor packing vents;
collect engine and compressor operational data;
b) an artificial intelligence model configured to:
analyze correlations between O2 fluctuations and manifold pressure instability;
analyze relationships between packing vent flow and pressure measurements;
analyze patterns of NOx and CO instability;
generate probability-based predictions of equipment failures;
c) a data collection system comprising:
sensors for measuring operational parameters;
an emissions analyzer;
a programmable logic controller for data synchronization.

2. The system of claim 1, wherein the emissions analyzer comprises an MKS FTIR analyzer configured to measure NOx, CH4, CO, and O2 concentrations in engine exhaust.

3. The system of claim 1, further comprising electrochemical sensors for measuring NOx, CO, and O2, and an NDIR sensor for measuring CH4.

4. The system of claim 1, further comprising a SCADA integration system configured to:
enable automated data collection from system sensors;
transmit collected data at configurable intervals;
integrate with facility-wide monitoring systems.

5. The system of claim 1, wherein the artificial intelligence model comprises a Long Short-Term Memory (LSTM) model hosted on a cloud platform.

6. The system of claim 1, further comprising a portable analyzer interface configured to enable facility technicians to perform AI analysis using portable electrochemical analyzers.

7. A method for predicting equipment failure in natural gas equipment, comprising:
a) collecting operational data including:
engine and compressor sensor data;
emissions data;
vent-gas measurements;
b) analyzing combined data patterns using an artificial intelligence model to:
identify correlations between O2 fluctuations and manifold pressure instability;
detect relationships between packing vent flow and pressure measurements;
monitor patterns of NOx and CO stability;
c) generating probability-based predictions of potential equipment failures based on the analyzed patterns.

8. The method of claim 7, further comprising synchronizing data collection between a vent-gas monitor and a compliance testing trailer.

9. The method of claim 7, further comprising transmitting collected data through a SCADA system at configurable intervals.

10. The method of claim 7, wherein generating probability-based predictions comprises calculating specific percentage probabilities of equipment failure within defined time periods.

11. A comprehensive monitoring system for natural gas equipment, comprising:
a) a fugitive gas capture system including:
collection points for multiple emission sources;
a filtration system;
an engine air intake system;
b) an artificial intelligence analysis system configured to:
analyze combined sensor and emissions data;
predict potential equipment failures;
generate probability-based assessments;
c) a data integration system configured to:
synchronize operational and emissions data;
enable periodic data transmission;
support portable analyzer integration.

12. The system of claim 11, wherein the artificial intelligence analysis system is configured to learn from historical failure data to improve prediction accuracy.

13. The system of claim 11, further comprising a compliance testing trailer with FTIR analyzers configured to interface with the fugitive gas capture system.

14. The system of claim 11, wherein the data integration system includes configurable transmission intervals for SCADA system integration.

15. The system of claim 11, further comprising permanent or temporary electrochemical and NDIR sensors for extended monitoring periods.
